(19) United States
(12) Patent Application Publication
(10) Pub. No.: US 2008/0045791 A1
(43) Pub. Date: Feb. 21, 2008

(54) Compact gynecological observation system for examination, imaging analysis and treatment

(76) Inventors: Ehud Gal, Reut (IL); Gennadiy Berinsky-Litiouga, Modi'in (IL); Ofer Pintel, Matan (IL)

Correspondence Address: William S. Frommer, Esq.;FROMMER LAWRENCE & HAUG LLP, 745 Fifth Avenue, New York, NY, 10151, US

(21) Appl. No.: 11/805,941

(22) Filed: May 25, 2007

Related U.S. Application Data
(63) Continuation of application No. PCT/IL05/01255, filed on Nov. 24, 2005.

(30) Foreign Application Priority Data
Nov. 30, 2004 (IL) 165482

(51) Int. Cl. A61B 1/303

(52) U.S. Cl. 600/116; 600/160

(57) ABSTRACT

The invention is a medical imaging device designed for insertion into the vagina for medical examination, imaging, image analysis and treatment of the interior of the vagina. The device comprises a front assembly, optimally shaped to match the shape of the vaginal canal; imaging and illumination assemblies; a handle; and an optimally shaped cover designed to fit tightly over and hermetically isolate at least that part of the front assembly that is inserted into the vaginal canal from the surrounding tissue and body fluids. The cover of the device of the invention can comprise one or more passages extending through it from its proximal end to its distal end enabling passage of one or more surgical tools and/or liquids and/or gases, from outside of the vaginal canal to the location of the scene imaged by the imaging assembly. The invention is also a medical imaging system comprising a medical imaging device of the invention, a computer and/or display station, and communication components. The medical imaging system may comprise operation software and image processing software.

### FIELD OF THE INVENTION

The present invention relates to the field of medical devices. Specifically, the invention relates to the field of medical devices for performing imaging, image analysis, and treatment. More specifically, the invention relates to a medical imaging device intended to be introduced into the vagina for medical examination, imaging, image analysis, and treatment.

### BACKGROUND OF THE INVENTION

Examination of the vaginal canal and cervix is a common procedure conducted by gynecologists world-wide on women of almost all ages. This procedure is conducted for detecting abnormalities in the vagina or cervix, including symptoms of cervical cancer and its pre-cancerous stages.

Cervical cancer is one significant abnormality in the vagina and cervix which requires detection since early detection of cervical cancer can bring to early treatment, which may result in healing of the cancer and saving the patient's life.

Additional abnormalities can be detected and identified by visual inspection of the vaginal canal. Such abnormalities include vaginal infections like fungus, bacterial vaginosis, parasite infections, and identification of sexually transmitted diseases and additional medical cases.

Observation and documentation of the vaginal canal of rape victims is necessary for detection of injuries as well as for treatment, documentation and evidence gathering.

In most gynecological examinations, it is not only required to see into the vaginal canal, it is also of great benefit to be able to capture and document images from inside the vagina for image processing, documentation for future reference, and for medical research and medical practice.

A significant element that is missing in existing gynecological examinations is the ability to “see and treat” at the same time, preferably using the same device. The implementation of surgical devices within a gynecological imaging device would enable viewing the vagina and cervix, and at the same time allow the medical practitioner to manipulate and operate surgical tools, while having a real-time view (and documentation) of the treatment procedure.

Medical practice of the prior art has failed to provide a comfortable way to carry out gynecological examinations. Until today, gynecological examinations are conducted while the patient is required to lie on her back on a gynecological treatment chair, with her legs spread open. This method of examination is not only uncomfortable, but is also considered by some as humiliating and many patients avoid their periodic gynecologic examination due to the nature of this procedure. In addition, most examinations require widening the vagina to enable the physician to perform the examination more thoroughly. In these cases a speculum is introduced into the vagina and is used to widen it. The constant advances in all aspects of medical practice bring a demand from both physicians and patients for more comfortable and less humiliating methods of examination.

As part of the attempt to discover cervical cancer and other vaginal abnormalities, a pap-smear is taken from the cervix and in some cases the physician performs biopsies. These samples are sent to a laboratory for further examination.

In cases where a standard gynecological examination or a pap-smear or a biopsy renders a troubling or inconclusive result, a colposcope, which is a lighted magnifying instrument used to examine the vagina and cervix from the exterior, is employed. Operation of this device also requires expanding the vagina with a speculum to enable clear view from the colposcope to the interior of the vagina. The use of the colposcope is uncomfortable for both the patient and the physician and often does not enable the physician to see clearly enough at all angles the lining of the vagina and the cervix.

It is the purpose of the present invention to provide a medical imaging device of compact proportions, which is intended for insertion into the vagina to enable on-line video imaging of the vaginal canal from inside.

It is another purpose of the present invention to provide a gynecological medical imaging device, which comprises a disposable cover, thereby eliminating the need for sterilization procedures.

It is another purpose of the present invention to provide gynecological medical imaging devices capable of acquiring an image having a field of view ranging from narrow to omni-directional.

It is further a purpose of the present invention to provide a medical imaging device, which enables documentation of part or the entire medical procedure.

It is yet another purpose of the present invention to provide a medical imaging device, which enables guidance of medical treatment tools through it enabling simultaneous performance of medical procedures while viewing the treatment area.

It is yet another purpose of the present invention to provide a medical imaging device complimented with software for control of the operation of the device as well as image processing and image understanding.

Additional purposes of the invention will become apparent as the description proceeds.

### SUMMARY OF THE INVENTION

In a first aspect, the invention is a medical imaging device designed for insertion into the vagina for medical examination, imaging, image analysis and treatment of the interior of the vagina. The device comprises:
a. a front assembly, optimally shaped to match the shape of the vaginal canal;
b. an imaging assembly comprising one or more image capture devices and associated optical lenses or lens systems, located in the front assembly and used to collect images of a scene located outside of the front assembly;
c. electronic components of the imaging assembly, including image transmission components, designed to transmit the image captured by the imaging assembly to a computer station and/or a display device;
d. a handle comprising operation and control switches and electronics;
e. an illumination assembly designed to illuminate the field of view covered by the imaging assembly;
f. an optimally shaped cover designed to fit tightly over and hermetically isolate at least that part of the front assembly that is inserted into the vaginal canal and the imaging assembly from the surrounding tissue and body fluids;
g. a front window, located on the cover, through which the imaging assembly is able to capture clear images of the scene and enabling the illumination assembly to illuminate the scene.

The cover of the device of the invention can comprise one or more passages extending through it from its proximal end to its distal end enabling passage of one or more surgical tools and/or liquids and/or gases, from outside of the vaginal canal to the location of the scene imaged by the imaging assembly. The cover may be disposable and detachable and can be replaced with a similar cover before each new procedure. The cover may cover the entire front assembly.

The optical lens of the device of the invention may be chosen from the following group:
a. a narrow field of view lens;
b. a fish-eye lens;
c. an omni-directional lens; and
d. a lens having zoom-in and zoom-out functions.

The image capture device may be capable of acquiring images still images and/or streaming video images. The images may be captured in at least one of the visible, near infrared, infrared, or ultraviolet spectral regions.

The image transmission components of the device may transmit the images by using any of wireless communication, transmission of optical signals through fiber optic cables, or transmission of electrical signals through wires or cables.

The illumination assembly of the device may Light Emitting Diodes located near the front window or an external illumination source, which generates light, which is conducted through the device to the window the illumination provided by the illumination assembly can be in at least one of the visible, near infrared, infrared, or ultraviolet spectral regions. Preferably the illumination is in substantially the same spectral range to which the image capture device is sensitive.

The passages in the cover of the device of the invention are designed to allow passage of one or more of the following:
a. medicinal fluids;
b. fluids for cleaning the interior of the vaginal canal;
c. fluids for cleaning the exterior of the cover;
d. gas for inflating inner spaces of the body;
e. surgical tools for collecting pap-smears; and
f. surgical tools for performing biopsies.

An embodiment of the medical imaging device of the invention comprises a balloon installed on the exterior of the cover. The balloon is connected to one of the channels such that pressurized gas supplied from outside of the device may be used to inflate the balloon. The expanding balloon pushes the vaginal walls away from the medical imaging device.

In another aspect, the invention is a medical imaging system. The system comprises:
a. a medical imaging device of the invention;
b. a computer and/or display station; and
c. communication components.

The medical imaging system of the invention may comprise operation software designed to control the operation of the imaging device, to capture images of interest and store them in a data base, and to control the zoom function of the optical lens.

The medical imaging system of the invention may also comprise image processing software designed to carry out one or more of the following operations:
a. to process the images acquired by the image capture device;
b. to detect abnormalities in an image acquired by the image capture device; and
c. to perform image comparison to detect changes between images taken at different locations, between images of the same scene captured at different occasions, or between an image and images in a data bank.

The communication components of the system of the invention are selected to provide communication between the medical imaging device and the computer and/or display station. They enable transmission of control commands from the control switches on the imaging device in order to control functions of the operation and/or image processing software and/or enable transmission of control commands from the operation and/or image processing software to control functions of the imaging device.

The control commands of the medical imaging system of the invention are transmitted by the communication components using wireless communication, transmission of optical signals through fiber optic cables, or transmission of electrical signals through wires or cables.

In yet another aspect the invention is a medical imaging device designed for insertion through the anus into the rectum for proctologic examination, imaging, image analysis and treatment of the rectum and of the anus. The device comprises:
a. a front assembly, optimally shaped to match the shape of the rectum;
b. an imaging assembly comprising one or more image capture devices and associated optical lenses or lens systems, located in the front assembly and used to collect images of a scene located outside of the front assembly;
c. electronic components of the imaging assembly, including image transmission components, designed to transmit the image captured by the imaging assembly to a computer station and/or a display device;
d. a handle comprising operation and control switches and electronics;
e. an illumination assembly designed to illuminate the field of view covered by the imaging assembly;
f. an optimally shaped cover designed to fit tightly over and hermetically isolate at least that part of the front assembly that is inserted into the rectum and the imaging assembly from the surrounding tissue and body fluids;
g. a front window, located on the cover, through which the imaging assembly is able to capture clear images of the scene and enabling the illumination assembly to illuminate the scene.

The cover comprises one or more passages extending through the cover from its proximal end to its distal end enabling passage of one or more surgical tools and/or liquids and/or gases, from outside of the anus to the location of the scene imaged by the imaging assembly. The cover can be disposable.

### BRIEF DESCRIPTION OF THE DRAWINGS

FIG. 1 shows the general structure of the medical device of the present invention;

FIG. 2 shows the disposable cover, which covers and protects the electro-optical assembly of the medical device;

FIG. 3 shows the medical device with the disposable cover in place;

FIG. 4 shows the channels in the disposable cover for guidance of treatment tools into the vagina;

FIG. 5 shows the entire medical device of the invention and one preferred method of its operation; and

FIG. 6 schematically shows the front electro-optical assembly of the medical device of the invention.

### DETAILED DESCRIPTION OF THE INVENTION

For a better understanding of the invention and to show how it may be carried into effect, reference will now be made, purely by way of example, to the accompanying drawings. With specific reference to the drawings in detail, it is stressed that the particulars shown are by way of example and for purposes of illustrative discussion of preferred embodiments of the present invention only, and are presented for the purpose of providing what is believed to be the most useful and readily understood description of the principles and conceptual aspects of the invention. In this regard, no attempt is made to show structural details of the invention in more detail than is necessary for a fundamental understanding of the invention. From the description taken together with the drawings it will be apparent to those skilled in the art how the several forms of the invention may be embodied in practice.

The device of the invention is designed to be introduced into the vagina for routine gynecological examinations as well as procedures such as collecting pap-smears, biopsy, irrigation and suction of liquids and/or gases into/from the vagina. Various obstetrical and infertility treatment procedures, e.g. objective dilatation measurement, under vision internal fetal heart rate monitoring electrode insertion, under-vision amniotomy, electro countering, presenting parts, embryo transfer, and intrauterine insemination can also be performed using the device of the present invention.

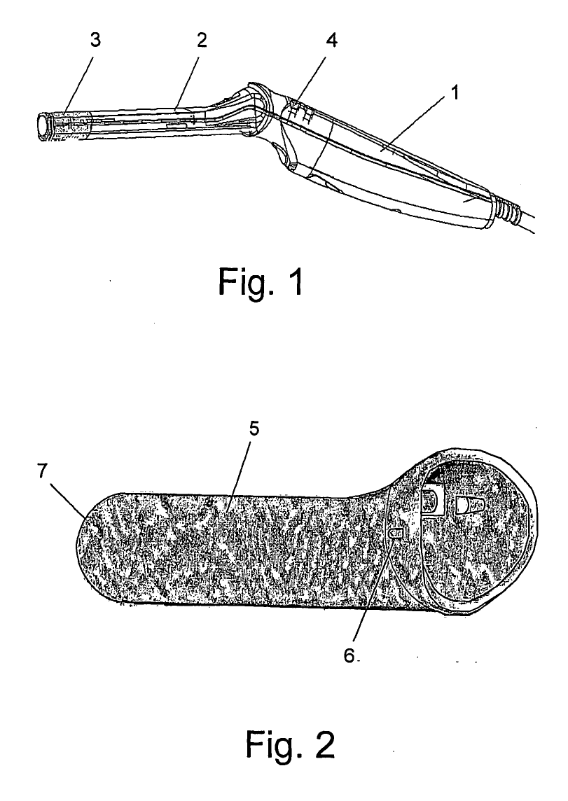

FIG. 1 shows schematically a perspective view of the medical device of the present invention. The device comprises a handle (1) and the front assembly (2). The front assembly is equipped with a forward viewing imaging assembly (3) designed to image the vaginal canal. All optical components and preferably all of the electronic components of the imaging assembly (3) are housed within the front assembly (2). The device is intended for introducing into the vaginal canal. Therefore the front assembly (2) is optimally shaped to match the shape of the vaginal canal to enable its introduction into the vagina with optimal comfort and ease.

On the handle (1), which always is outside of the vaginal canal, are located operation and control switches (4). The operation and control electronics are housed within the handle (1). Wires from the operation and control electronics, power cords and image output wires (not shown) are housed within the handle (1) and the front assembly (2).

The medical procedure of examining the vaginal canal is performed by introducing the front assembly (2) of the device into the vagina, while the physician holds the handle (1) to guide the device and control the device's operation by means of the control switches (4) located on the handle (1).

It is possible to equip the device with switches (4) having many different purposes, depending on the requirements of the procedure. Some typical, but non-limitative, examples are:
an on/off switch for the device;
a release and lock button for the disposable cover if one is present;
switches that control the operation of treatment tools;
an image capture switch, to enable capture and documentation of selected images during the procedure; and
switches that control the software interface of the device.

The image acquired by the imaging assembly (3) can be transmitted to a display station only or preferably to a computer station capable of providing control, image processing, and display functions. The images are presented on the screen and image processing functions may be applied to the images for various purposes. The signals, either optical or electrical, are transferred from the device to the external display/computing unit via wires or cables, extending from the rear of the handle, or by means of wireless communication devices.

Since this medical device is intended for repeated use, it is essential to maintain appropriate sterility of the front assembly (2), which is inserted into the vagina. For this purpose it is possible to perform standard sterilization procedures on the front assembly (2), however, it is also possible to utilize a disposable cover on the front assembly. The use of a disposable cover is preferable because it eliminates the expense and “down time” of the device that are required for sterilization. Furthermore, the materials and procedures used for sterilization of medical devices generally cause deterioration of the device, especially its optical and electronic components, with time. The number of sterilization procedures that a device can undergo is dependent on the materials of which it is comprised and therefore has a significant influence on its cost.

FIG. 2 shows one embodiment of a disposable cover (5) designed to hermetically isolate the front assembly (2) and the imaging assembly (3) (shown in FIG. 1) from the surrounding tissue and body fluids. The disposable cover (5) is designed to be replaced between patients, such that each patient may undergo an examination using sterile equipment. The disposable cover (5) fits over the entire front assembly (2) and can be connected to it by various methods. One illustrative method of connecting the disposable cover to the front assembly is to provide slots (6) in the cover (5), such that they interlock with corresponding clips (not shown in the figures) on the sides of the front assembly (2). In this embodiment a Release/Lock button/lever, which enables the tight attachment of the disposable cover to the front assembly and its quick release, can be provided on handle (1).

The disposable cover is designed to cover the imaging assembly (3), which, in the embodiment shown, is located at the front of the front assembly (2) and is oriented to gather a forward looking view. In order to prevent deterioration in the image quality caused by the use of a cover over the imaging assembly (3), it is important to maintain maximum optical transparency of the front window (7) of the disposable cover (5). The front window (7) is defined as the part of the disposable cover (5), which is between the imaging assembly (3) and the field of view which is to be covered by it.

Although the description hereinabove refers to a “disposable cover”, the primary purpose of the cover is to cover the outer surface of the front assembly and thereby to remove the necessity of sterilizing the medical device itself between patients. This distinction is made to indicate that it is recommended to maintain a replaceable (preferably “disposable”) component between the medical device and body tissue. It is obvious that the cover may be detached from the device, be sterilized, and then re-attached for repeated use. Therefore the term “disposable”, as used herein with reference to the cover, may refer to a cover made for either single or multiple uses.

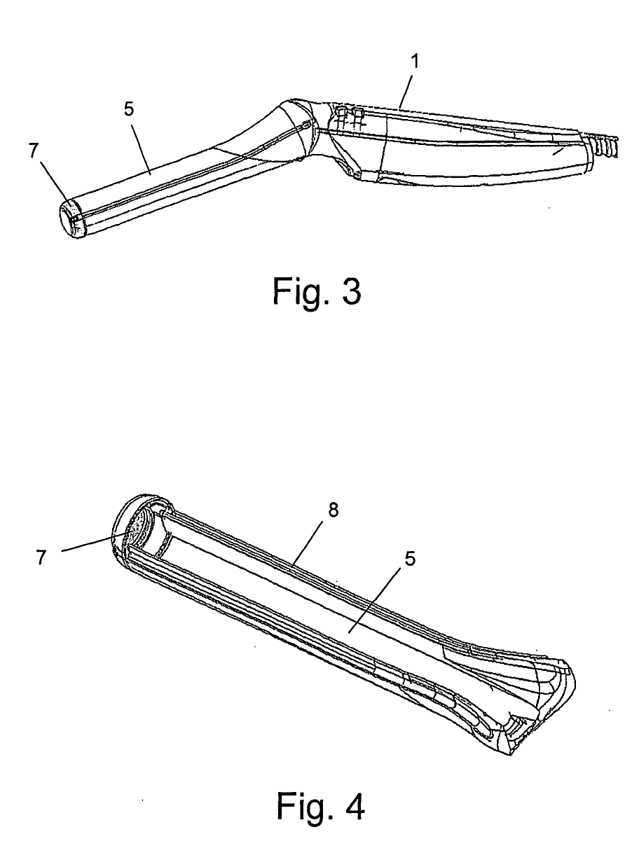

FIG. 3 shows the medical device of the present invention with the disposable cover (5) attached to it covering the front assembly and imaging assembly of the device. The disposable cover (5) is equipped with an optically transparent window (7), through which the imaging assembly may acquire the image with minimal negative effect on the quality of the image. In some embodiments, the window (7) may have optical qualities that enhance or intensify image quality, e.g. the window may be designed to optically magnify the image, to filter specific wavelengths, etc.

It is stressed that both the front assembly (2) as shown in respect to FIG. 1 and the disposable cover (5) that fits over it are optimally shaped to fit the shape of the vaginal canal in order to penetrate with optimal ease and comfort. It is obvious that the device in its entirety, with particular emphasis on the front assembly (2) and the disposable cover (5), may be designed and manufactured to fit patients of different ages and having different anatomical proportions in order to maintain the ease and comfort that accompanies the use and insertion of the device into the vagina.

During gynecological examinations it is sometimes required to take therapeutic measures, or to carry out procedures beyond simple inspection. These procedures may include: taking a pap-smear or biopsy of the vaginal canal; inflating or washing the vaginal canal; administering fluids having medicinal affects to the vagina; extracting foreign objects; performing suction, etc. There is a great benefit in terms of saving of time and effectiveness of treatment to be gained from being able to view the vagina from within while performing these procedures. To be able to carry out a medical procedure while simultaneously viewing the treatment area in the interior of the vagina, embodiments of the invention are provided with means for guiding tools, fluids and/or gases from the exterior into the vagina (and vice versa), while the device is in place inside the vagina and is used to monitor the procedure.

FIG. 4 shows a partial longitudinal cross sectional view of the disposable cover (5). In this figure, guidance channels (8), through which medical tools, fluids and/or gases for treatment and operation inside the vagina and in the area of the cervix can pass, are created in or on the walls of disposable cover (5). The tools, fluids or gases are pushed, or caused to flow from the exterior, through the guidance channels (8) and exit from the front side of the disposable cover (5), adjacent to the optical window (7), such that it is possible to view them and the results achieved by using them with the imaging assembly, thorough the optical window (7).

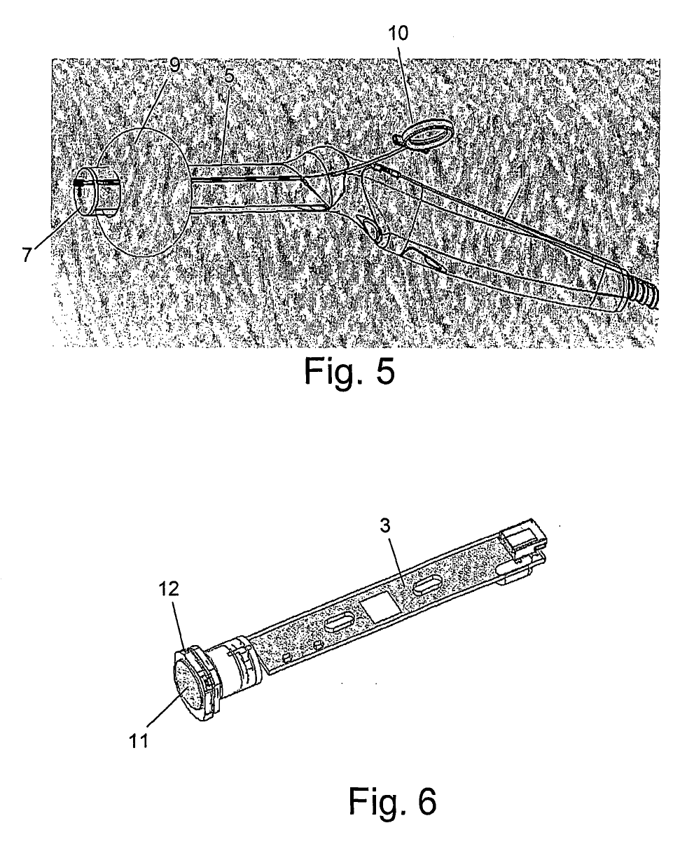

FIG. 5 shows an embodiment of the device of the invention comprising a biopsy tool and an inflatable balloon. In this figure the disposable cover (5) is attached to the handle (1) of the device. An inflatable balloon (9) is installed on the exterior of the disposable cover (5) and connected to one of the guidance channels (8) such that it is possible to inflate it by means of compressed air supplied from a source connected to the handle (1) of the device and flowing through a control switch 4 and the guidance channel (8) in the wall of the disposable cover (5). The purpose of the inflatable balloon (9) is to push the walls of the vagina away from the device when it is inserted into the vagina in order to enable an unobstructed view or to enable easier of greater maneuverability of the treatment tools inside the vagina. A treatment tool (10), for example a biopsy tool, is pushed through another channel of the disposable cover, from the handle (1) in the direction of the area at which the procedure is to be carried. Tool (10) exits from the front of the disposable cover (5) adjacent to the optical window (7), which enables the imaging assembly to view the treatment area. The treatment tool is operated from the exterior and is pushed back and forth and rotated as necessary by the physician.

There can be provided embodiments of the invention comprising several channels in the disposable cover (5) to enable the use of several tools at the same time, each tool passing through a separate channel. It is also possible to use some of the channels for tools while others may be used for gases and/or liquids. Fluids may be used to wash the vaginal canal from within or to wash the tools or the optical window (7) on the disposable cover (5). Preferably one of the channels is fitted with a nozzle at its distal tip so that water or air can be sprayed on the outside of the window to keep it clean. As mentioned hereinabove, it is also possible to use fluids to produce medicinal affects (drug delivery

Although the description of the guidance channels hereinabove refers to channels in the disposable cover, it is obvious that if a disposable cover is not implemented, and the front assembly is designed to come in direct contact with the body, then guidance channels may be implemented in the interior of the front assembly.

FIG. 6 schematically shows the electro-optics assembly, which is located at the front of the medical device. The imaging assembly (3) includes an image capture device (not shown), an optical lens or lens system (11), and preferably an illumination assembly (12). It is possible to use any type of image capture device, still or video, which fits into the dimensions of the medical device. Alternatively, it is possible to use image capture devices, which have the image sensor positioned remotely from the rest of the electronics, thus the image sensor fits into the front of the device (i.e. the part that is to enter into the vagina) and the rest of the electronics is remote (for example, in the handle of the device). Another possibility is to have the image capture device, including the image sensor, remote (i.e. close to the handle side) and to use rod lenses or fiber optics to conduct the image from the front assembly to the image capture device. The image capture device may be such that it is sensitive to a selected spectral range (i.e. visible, IR, NIR, UV etc.) in order to enable detection of different types of image data which are visible at certain wavelengths. The optical lens (11) which is connected to the image capture device is preferably a wide angle lens, such that it is capable of acquiring an image of 120 degrees or more. Such lenses are known as “Fish-Eye” lenses. Alternatively it is possible to utilize omni-directional lenses which are based on manipulation of reflective and refractive surfaces to capture a field of view of more than 180 degrees. Additionally, if necessary, the optical system can be designed to have a narrow field of view or with zoom capabilities in order to obtain higher image quality or “close-up” views of particular regions.

The images are transmitted either through cables or wirelessly by the transmission components of the imager and presented on a screen enabling the physician as well as the patient to experience a more convenient and less humiliating examination procedure. Preferred embodiments of the invention come with either a dedicated or a standard operation software package to provide application interface, image processing tools, and additional computerized functions using for example a personal computer, which can also be used to store the images for documentation of part or the entire medical procedure. One example of the use of such software would be the comparison of images, either from different locations, from previous examinations, or from a data bank for the detection of abnormal clusters, formations, and colors in the vagina and cervix.

An illumination assembly (12) is also located in the front of the imaging assembly and it is designed to illuminate the field of view, which is to be captured by the image capture device. It is possible to use an illumination assembly which can illuminate with a selected spectral range, such that is suitable to that to which the image capture device is sensitive. It is also possible to use an illumination source which combines several illumination modules each capable of illuminating the area with light from different spectral regions. The illumination assembly can be comprised of elements such as photodiodes attached to or adjacent to the optical window (7) of the cover (5) and supplied, by means of wires passing through one or more channels (8) in cover (5), with energy from a source located in the handle or external to the device of the invention. Alternatively, the light source is located in the handle or external to the device and the light is guided to the window (7) by means of optical fibers passing through one or more channels (8) in the cover (5).

It is stressed that while the description herein refers to a forward-viewing imaging assembly, it is also possible to utilize any other configuration as may be needed. The example of a forward viewing imaging system is particularly relevant, for example, for viewing the cervix and its vicinity. Other medical procedures may require viewing the perimeter or the walls of the vagina and for this purpose a side-viewing imaging assembly might be more suitable. It is also possible to fit other combinations of image sensors and optical assemblies in the device of the invention to enable combined forward-viewing with side-viewing image sensors, image sensors gathering images from two sides of the front assembly, etc.

Although embodiments of the invention have been described by way of illustration, it will be understood by persons familiar with the art that the invention may be carried out with many variations, modifications, and adaptations. For example, a medical imaging device very similar to the one of the invention could be built mutandis mutatis for insertion through the anus into the rectum for use in carrying out proctologic examinations and medical procedures.

1. A medical imaging device designed for insertion into the vagina for medical examination, imaging, image analysis and treatment of the interior of the vagina, said device comprising:
a. a front assembly, optimally shaped to match the shape of the vaginal canal;
b. an imaging assembly comprising one or more image capture devices and associated optical lenses or lens systems, located in said front assembly and used to collect images of a scene located outside of said front assembly;
c. electronic components of said imaging assembly, including image transmission components, designed to transmit the image captured by the imaging assembly to a computer station and/or a display device;
d. a handle comprising operation and control switches and electronics;
e. an illumination assembly designed to illuminate the field of view covered by said imaging assembly;
f. an optimally shaped cover designed to fit tightly over and hermetically isolate at least that part of said front assembly that is inserted into said vaginal canal and said imaging assembly from the surrounding tissue and body fluids;
g. a front window, located on said cover, through which said imaging assembly is able to capture clear images of said scene and enabling said illumination assembly to illuminate said scene.

2. A medical imaging device according to claim 1 comprising a cover having one or more passages extending through the cover from its proximal end to its distal end enabling passage of one or more surgical tools and/or liquids and/or gases, from outside of the vaginal canal to the location of the scene imaged by the imaging assembly.

3. A medical imaging device according to claim 1, wherein the cover is disposable.

4. A medical imaging device according to claim 1, wherein the cover covers the entire front assembly.

5. A medical imaging device according to claim 1, where the cover is detachable and can be replaced with a similar cover before each new procedure.

6. A medical imaging device according to claim 1, wherein the optical lens is chosen from the following group:
a. a narrow field of view lens;
b. a fish-eye lens;
c. an omni-directional lens; and
d. a lens having zoom-in and zoom-out functions.

7. A medical imaging device according to claim 1, wherein the image capture device is capable of acquiring images selected from the group comprising:
a. still images; and
b. streaming video images.

8. A medical imaging device according to claim 1, wherein the image capture device is capable of acquiring images in at least one of the visible, near infrared, infrared, or ultraviolet spectral regions.

9. A medical imaging device according to claim 1, wherein the image transmission components transmit the images by using one or more of the following technologies:
a. wireless communication;
b. transmission of optical signals through fiber optic cables; and
c. transmission of electrical signals through wires or cables.

10. A medical imaging device according to claim 1, wherein the illumination assembly comprises a light source chosen from the group comprising:
a. Light Emitting Diodes located near the front window; and
b. an external illumination source, which generates light, which is conducted through said device to the window.

11. A medical imaging device according to claim 1, wherein the illumination assembly provides illumination in at least one of the visible, near infrared, infrared, or ultraviolet spectral regions.

12. A medical imaging device according to claim 1, wherein the illumination assembly provides illumination in substantially the same spectral range to which the image capture device is sensitive.

13. A medical imaging device according to claim 2, wherein the passages in the cover are designed to allow passage of one or more of the following:
a. medicinal fluids;
b. fluids for cleaning the interior of the vaginal canal;
c. fluids for cleaning the exterior of the cover;
d. gas for inflating inner spaces of the body;
e. surgical tools for collecting pap-smears; and
f. surgical tools for performing biopsies.

14. A medical imaging device according to claim 2, comprising a balloon installed on the exterior of the cover and connected to one of the channels such that pressurized gas supplied from outside of said device may be used to inflate said balloon thereby pushing the vaginal walls away from said medical imaging device.

15. A medical imaging system comprising:
a. a medical imaging device according to claim 1;
b. a computer and/or display station; and
c. communication components.

16. A medical imaging system according to claim 15, comprising operation software designed to carry out one or more of the following operations:
a. to control the operation of the imaging device;
b. to capture images of interest and store them in a data base; and
c. to control the zoom function of the optical lens.

17. A medical imaging system according to claim 15, comprising image processing software designed to carry out one or more of the following operations:
a. to process the images acquired by the image capture device;
b. to detect abnormalities in an image acquired by the image capture device; and
c. to perform image comparison to detect changes between images taken at different locations, between images of the same scene captured at different occasions, or between an image and images in a data bank.

18. A medical imaging system according to either of claims 15, wherein the communication components are selected to provide communication between the medical imaging device and the computer and/or display station; wherein said communication components enable transmission of control commands from the control switches on said imaging device in order to control functions of the operation and/or image processing software and/or enable transmission of control commands from said operation and/or image processing software to control functions of said imaging device.

19. A medical imaging system according to claim 18, wherein the control commands are transmitted by the communication components using one or more of the following technologies:
a. wireless communication;
b. transmission of optical signals through fiber optic cables; and
c. transmission of electrical signals through wires or cables.

20. A medical imaging device designed for insertion through the anus into the rectum for proctologic examination, imaging, image analysis and treatment of the rectum and of the anus, said device comprising:
a. a front assembly, optimally shaped to match the shape of the rectum;
b. an imaging assembly comprising one or more image capture devices and associated optical lenses or lens systems, located in said front assembly and used to collect images of a scene located outside of said front assembly;
c. electronic components of said imaging assembly, including image transmission components, designed to transmit the image captured by the imaging assembly to a computer station and/or a display device;
d. a handle comprising operation and control switches and electronics;
e. an illumination assembly designed to illuminate the field of view covered by said imaging assembly;
f. an optimally shaped cover designed to fit tightly over and hermetically isolate at least that part of said front assembly that is inserted into said rectum and said imaging assembly from the surrounding tissue and body fluids;
g. a front window, located on said cover, through which said imaging assembly is able to capture clear images of said scene and enabling said illumination assembly to illuminate said scene.

21. A medical imaging device according to claim 20 comprising a cover having one or more passages extending through the cover from its proximal end to its distal end enabling passage of one or more surgical tools and/or liquids and/or gases, from outside of the anus to the location of the scene imaged by the imaging assembly.

22. A medical imaging device according to claim 20, wherein the cover is disposable.
